(19) United States
(12) Patent Application Publication
(10) Pub. No.: US 2025/0339318 A1
(43) Pub. Date: Nov. 6, 2025

(54) Saturation Detection in an Absorbent Article

(71) Applicant: Raja Singh Tuli, Montreal (CA)

(72) Inventor: Raja Singh Tuli, Montreal (CA)

(21) Appl. No.: 19/196,442

(22) Filed: May 1, 2025

Related U.S. Application Data
(60) Provisional application No. 63/641,668, filed on May 2, 2024.

(51) Int. Cl. A61F 13/42

(52) U.S. Cl. CPC A61F 13/42; A61F 2013/424

(57) ABSTRACT

An adjustable threshold is used to determine the saturation of an absorbent article, in order to improve the accuracy. The adjustable threshold may be adjusted based on the tightness/looseness of the absorbent article on its wearer and/or based on the motion/movement of the wearer of the absorbent article wearer.

### CROSS-REFERENCE TO RELATED APPLICATIONS

This application claims priority to U.S. provisional patent application No. 63/641,668, entitled “Saturation Detection in an Absorbent Article,” filed on May 2, 2024. The content of this U.S. provisional patent application is hereby incorporated by reference in its entirety for all purposes.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

### Field of the Invention

The present disclosure relates to systems and methods for saturation detection in an absorbent article, in particular to the threshold adjustment for saturation detection in an absorbent article.

### Description of Related Art

For many years, a variety of designs have been developed for detecting and signaling the wetness and/or saturation in an absorbent article such as a diaper. However, the accuracy of the wetness and/or saturation detection is not satisfactory and/or remains doubtful. Therefore, there is a need to improve the accuracy of the wetness and/or saturation detection.

Furthermore, in the prior art, there is no disclosure that takes into account factors such as the tightness/looseness of an absorbent article and/or the motion/movement of the absorbent article wearer and/or the orientation of the absorbent article when determining the saturation of the absorbent article.

### BRIEF SUMMARY OF THE INVENTION

Many care facilities have no efficient way to determine, monitor, and schedule service and visits based on the real time needs of the patient. Patients are often left in their own urine and feces for extended periods of time, which may cause health problems. This leads to an increased demand for alternative incontinence management solutions. In accordance with the present subject matter, we have invented a sensor system for monitoring incontinence in patients/residents and for facilitating timely attention to resident needs in an institutional setting. This system not only facilitates things for the residents, but also for their caregivers too.

In a preferred embodiment of the present disclosure, there may be at least two carbon lines along the length of e.g. an impermeable layer of an absorbent article (such as but not limited to diapers, briefs, under pads, fitted briefs, belted shields, liners, all-in-one pads, pull-up incontinence pants, protective underwear). For the purpose of clarity, in this application, a diaper will be used to illustrate the possible embodiments of the disclosure. In the healthcare context, a diaper is an absorbent pad made of one or more various materials and used for personal hygiene.

In an aspect of the present disclosure, a method for detecting the saturation of an absorbent article is provided. The method comprises detecting a sense signal from the absorbent article, and comparing the detected sense signal to a saturation threshold. According to an embodiment of the present disclosure, the saturation threshold is adjustable.

According to an embodiment of the present disclosure, the saturation threshold may be automatically adjusted based on tightness of the absorbent article on its wearer's body. In an implementation, the method may further comprise determining the tightness of the absorbent article on its wearer's body, and adjusting the saturation threshold based on the determined tightness.

According to another embodiment of the present disclosure, the saturation threshold may be automatically adjusted based on amount of the movement of the wearer of the absorbent article. In an implementation, the method may further comprise determining the movement of the wearer of the absorbent article during a certain time period after a wetness event, and adjusting the saturation threshold based on the determined movement of the wearer. Preferably, the adjusting the saturation threshold may comprise: continuously adjusting the saturation threshold based on the total amount of movements of the wearer of the absorbent article over time during the certain time period after the wetness event. Alternatively, the adjusting the saturation threshold may comprise: continuously adjusting the saturation threshold based on the weighted sum of movements of the wearer of the absorbent article over time during the certain time period after the wetness event. The weight for the weighted sum may decrease over time. In a particular implementation, the movement may be determined based upon the change in orientation of the absorbent article measured periodically. Alternatively, the movement of the wearer of the absorbent article may be detected by a movement detector provided in an attachment unit that is configured to be attached to the absorbent article and to apply the drive signal and sense the sense signal.

According to a further embodiment of the present disclosure, the saturation threshold may be automatically adjusted based on the change in the sense signal detected after the end of a wetness event in the absorbent article. In an implementation, the end of the wetness event may be determined based on the start of the wetness event and one of a predetermined fixed time interval or the inflection point of the sense signal after the start of the wetness event.

In another aspect of the present disclosure, a method for detecting a wearing state of an absorbent article on its wearer is provided. The method comprises: determining a sense signal from the absorbent article after the end of a wetness event in the absorbent article; and determining the wearing state of the absorbent article on its wearer based on the determined sense signal. In an implementation, the absorbent article may be determined as being tight on its wearer when the determined sense signal continues to increase after the end of the wetness event. The end of the wetness event may be determined based on the start of the wetness event and one of a predetermined fixed time interval or the inflection point of the sense signal after the start of the wetness event.

### BRIEF DESCRIPTION OF THE DRAWINGS

The various preferred embodiments of the present disclosure described herein can be better understood by those skilled in the art when the following detailed description is read with reference to the accompanying drawings. The components in the figures are not necessarily drawn to scale and any reference numeral identifying an element in one drawing will represent the same element throughout the drawings. The figures of the drawing are briefly described as follows.

FIG. 1 illustrates an exemplary absorbent article in an exploded perspective view according to an embodiment of the present disclosure;

FIG. 2 illustrates an exemplary absorbent article with four spaced-apart conductive lines being provided on the top side of its substantially liquid impervious layer according to an embodiment of the present disclosure;

FIG. 3 illustrates an exemplary attachment unit (e.g. pod) that is configured to be attached to a disposable absorbent article and to operate in combination with the spaced-apart conductive lines in the absorbent article for moisture detection and estimation of the absorbent article, according to an embodiment of the present disclosure;

FIG. 4 schematically depicts wetness events in an absorbent article, according to an embodiment of the present disclosure;

FIG. 5 schematically illustrates a sense signal sensed from a sense electrode (e.g. one of the conductive lines) in an absorbent article for wetness events, according to an embodiment of the present disclosure;

FIG. 6 schematically illustrates two example wearing states/manner of an absorbent article, a loose state on wearer and a tight state on wearer, according to an embodiment of the present disclosure;

FIG. 7 schematically illustrates an example development of the urine spot for an urination in an absorbent article, according to an embodiment of the present disclosure; and

FIG. 8 schematically illustrates the example signal sensed from the absorbent article for the urination as illustrated in FIG. 7, according to an embodiment of the present disclosure.

FIG. 9 schematically illustrates how to determine a baseline and how to adjust threshold based on the baseline, according to an embodiment of the present disclosure.

FIG. 10 schematically illustrates two example wetness (e.g. urination) spots produced when a wetness event occurs with different angles in relative to an absorbent article, according to an embodiment of the present disclosure.

FIG. 11 schematically illustrates an example movement of an absorbent article's wearer from lying down to sitting up, according to an embodiment of the present disclosure.

While the features described herein are susceptible to various modifications and alternative forms, specific embodiments thereof are shown by way of example in the drawings and are herein described in detail. It should be understood, however, that the drawings and detailed description thereto are not intended to be limiting to the particular form disclosed, but on the contrary, the intention is to cover all modifications, equivalents and alternatives falling within the spirit and scope of the subject matter as defined by the appended claims.

### DETAILED DESCRIPTION OF THE INVENTION

Disposable absorbent article such as disposable diaper is a product that is capable of receiving and retaining bodily exudates or excretions so as to prevent contamination of the clothing or external environment. As an example, with a disposable diaper, the user is allowed to urinate or defecate without the use of a toilet. In addition to diapers, there are numerous other types of disposable absorbent articles such as e.g. under pads, incontinence pads, fitted briefs, belted shields, liners, all-in-one pads, pull-up incontinence pants, training pants, protective underwear, catamenial napkins, and incontinence guards etc. It is to be understood that the list of disposable absorbent articles identified above is not exhaustive and that these and other absorbent articles can be used with the present disclosure and are within the scope of the present disclosure. It is also to be understood that a reference in this specification to any one such article, such as a “diaper” is to be taken to be a reference to any and all other suitable absorbent articles including incontinence garments, pads and the like.

In order to prevent contamination of the clothing or external environment, disposable absorbent article is provided with an absorbent core capable of receiving and retaining bodily exudates or excretions, and a substantially liquid impervious layer. In general, disposable absorbent products consist of a layered construction, which allows the bodily exudates or excretions to be distributed and transferred to the absorbent core where they are retained in. In everyday use, a disposable absorbent article may be used until the absorbent core is saturated with e.g. bodily exudates or excretions. When the absorbent core is saturated, the disposable absorbent article needs to be removed, disposed of, and replaced with a clean and dry article.

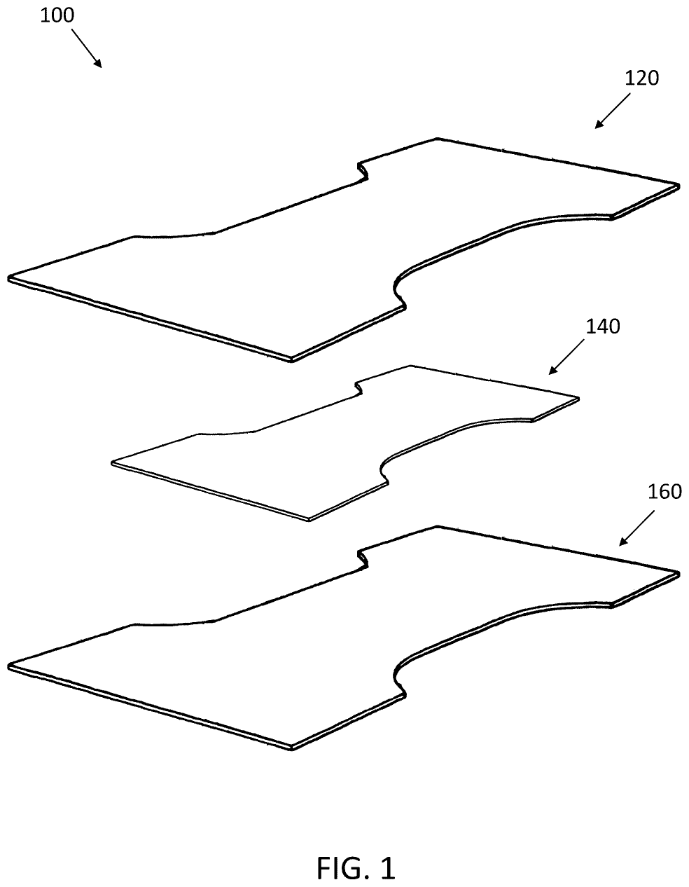

FIG. 1 illustrates an exemplary disposable absorbent article in an exploded perspective view. As illustrated, a disposable absorbent article 100 primarily consists of an absorbent core 140 sandwiched between a liquid pervious layer 120 and a substantially liquid impervious layer 160.

As illustrated in the exemplary diaper of FIG. 1, a disposable absorbent article 100 has a substantially liquid impervious layer 160 configured to prevent the bodily exudates or excretions absorbed and retained in the absorbent core 140 from wetting articles, such as bed sheets and undergarments, which contact the disposable absorbent article 100. On top of the layer 160 is disposed an absorbent core 140 made of a superabsorbent material. On top of the absorbent core 140 is a liquid pervious layer 120 that is joined to the layer 160 in an assembled state of the disposable absorbent article and is placed next to the skin of the user when in use. Additional structural features such as additional layer(s), elastic members and fastening means for securing the article in place, such as tape tab fasteners, may also be included.

The liquid pervious layer 120 is configured to be penetrable by bodily exudates and excretions in a direction into the absorbent core 140 to enable them to be absorbed and retained in the underlying absorbent core 140. It is appreciated that the layer 120 may be made of a variety of liquid pervious materials, e.g. nonwoven fabric.

The absorbent core 140 may be made up of hydrophilic superabsorbent polymers (SAP) and fibrous material, as a non-limiting example. The polymers act like tiny sponges that retain many times their weight in liquid.

The substantially liquid impervious layer 160 is made of a material substantially impervious to liquids. As an example, the substantially liquid impervious layer 160 may be manufactured from a thin plastic film, although other liquid impervious materials may also be used. As described above, the substantially liquid impervious layer 160 is configured to prevent the bodily exudates or excretions absorbed and retained in the absorbent core from wetting articles, such as bed sheets and undergarments, which contact the diaper.

As illustrated in the exemplary diaper of FIG. 1, the layers 120 and 160 are coextensive and have generally larger dimension, in length and/or width, than the absorbent core 140.

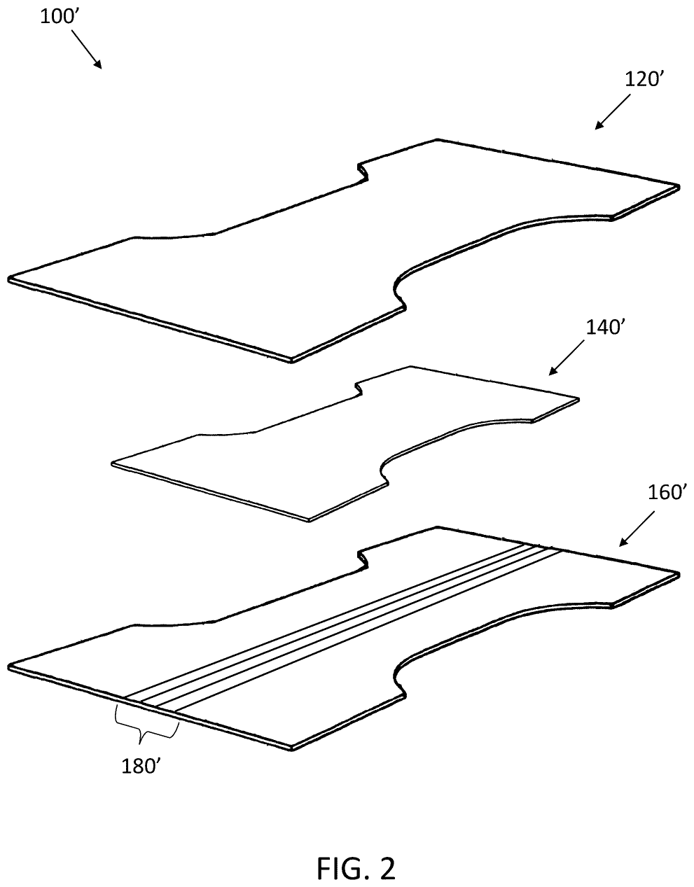

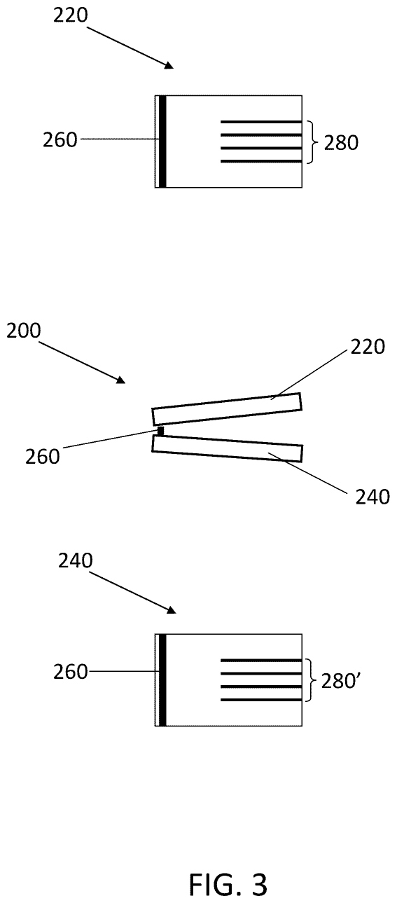

In order for moisture detection and estimation, in particular to detect the presence and/or amount and/or saturation of the bodily exudates or excretions in an absorbent article, in particular in its absorbent core, a number of (e.g. at least two) spaced-apart conductive lines may be provided as electrodes on e.g. the top side (i.e. the side facing the absorbent core) of the substantially liquid impervious layer along the length of the absorbent article, in an embodiment of the present disclosure. In FIG. 2, an exemplary disposable absorbent article 100′ is depicted with four spaced-apart conductive lines 180′ being provided on the top side of the substantially liquid impervious layer 160′, as an example. The spaced-apart conductive lines 180′ in the disposable absorbent article 100′ operate in cooperation with an attachment unit e.g. pod 200 (to be described below in reference to FIG. 3), for moisture detection and estimation of the absorbent article 100′.

It is to be noted that, in a disposable absorbent article the spaced-apart conductive lines may be provided on any layer, e.g. on the top side of the substantially liquid impervious layer 160′ as illustrated in FIG. 2, or on any side of the liquid pervious layer 120 or of the absorbent core 140 or even embedded in the absorbent core 140 as illustrated in FIG. 1. In a primary embodiment, the spaced-apart conductive lines are provided on one of the bottom layers, such as the bottommost layer, e.g. the substantially liquid impervious layer, in an absorbent article.

According to an embodiment of the present disclosure, an attachment unit (e.g. pod) is configurable to be attached (preferably, releasably attached) to a disposable absorbent article and to operate in combination with the spaced-apart conductive lines in the disposable absorbent article for moisture detection and estimation of the absorbent article. As illustrated in FIG. 3, an exemplary attachment unit e.g. pod 200 primarily consists of two halves 220 and 240 that are pivotably coupled to each other with a pivotal connection 260. At least one of the two halves 220 and 240 is provided with a number of (e.g. at least two) contacts 280 or 280′ on its inner side (i.e. the side facing the other half). As a non-limiting example, both the two halves 220 and 240 have each a number of contacts 280 or 280′ on their respective inner side, as illustrated in FIG. 3. In the example as illustrated in FIG. 3, there are four (the same number as the conductive lines 180′ as illustrated in FIG. 2) contacts 280 and 280′ on the two halves 220 and 240 respectively.

In operation, the pod 200 as illustrated in FIG. 3 is clipped on a disposable absorbent article (e.g. 100′ as illustrated in FIG. 2) at one of its waist end edges and coupled to the conductive lines (e.g. 180′ in FIG. 2) with the contacts 280 and/or 280′ on the pod 200. It is to be noted that, the coupling between the conductive lines in an absorbent article and the contacts on an attachment unit may be electrical, capacitive, or resistive coupling. In use, the bodily exudates or excretions absorbed and retained in the absorbent core (e.g. 140′ in FIG. 2) of the absorbent article will cause at least two of the spaced-apart electrodes (conductive lines, e.g. 180′ in FIG. 2) to be connected to each other, and thus the pod 200 can detect the presence and/or amount of the exudates or excretions in the disposal absorbent article (e.g. 100′ in FIG. 2) by e.g. applying a drive signal to at least one of the electrodes (the drive electrode) and sensing a sense signal from at least another one of the electrodes (the sense electrode).

As describe above, in a disposable absorbent article the spaced-apart conductive lines may be provided on any layer, on which at least two of the spaced-apart conductive lines might be connected to each other with the aid of the bodily exudates or excretion absorbed and retained in the disposable absorbent article, which in turn enables the detection of the presence and/or amount and/or saturation of the exudates or excretions in the disposal absorbent article.

Next, the wetness and/or saturation detection in an absorbent article will be described with reference to an absorbent article with two parallel conductive lines being provided along its length, as a non-limiting example. In use, a drive or input signal (e.g. AC signal or DC signal such as a voltage) may be applied to one of the conductive lines, and a sense or output signal (e.g. a current) may be sensed from another one of the conductive lines. The wetness and/or saturation in the absorbent article such as the diaper may be determined based on the output signal as sensed.

In particular, an attachment unit such as a pod e.g. that as illustrated in FIG. 3 is placed (e.g. clipped) on an absorbent article such as a diaper (e.g. on its front waist portion) with the contacts on the unit being properly aligned with the conductive lines on the absorbent article such as the diaper either with physical contact or with capacitive contact. The unit applies a drive signal e.g. a voltage to one of the conductive lines in the diaper (drive line), and senses a sense signal e.g. a current from another one of the conductive lines (sense line). Based on the sense signal sensed from the sense line, it is possible to determine the wetness event and/or the amount of liquid and/or the saturation in the diaper.

In use, a user wears an absorbent article e.g. a diaper with an attachment unit placed thereon. When a first wetness event e.g. a first urination occurs (which lasts e.g. about 10 seconds), the liquid e.g. urine hits the diaper and forms at a first location a first closed circuit with the unit, the drive line and the sense line, and then the liquid goes further out and expands in both directions from the first location, and finally ends in a second closed circuit between the drive line and the sense line at a second location.

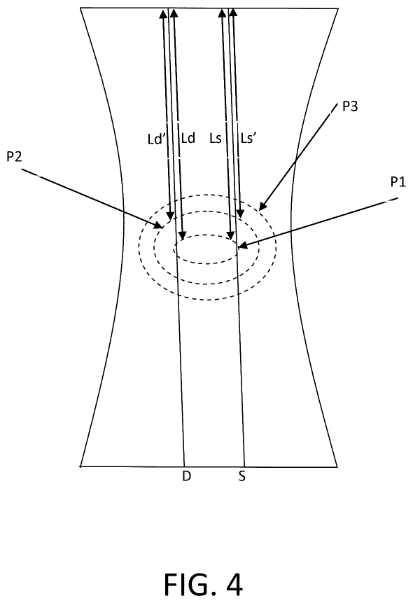

FIG. 4 schematically depicts wetness events in an absorbent article (e.g. a diaper) with two conductive lines (as electrodes), according to an embodiment of the present disclosure. As depicted in FIG. 4, when a first wetness event occurs in an absorbent article, the liquid hits the absorbent article and forms a first closed circuit between the two conductive lines, i.e. the drive line D and the sense line S, at a first location P1, and ends in forming a second closed circuit between the drive line D and the sense line S at a second location P2 as the liquid expands in the diaper. It is to be noted that the length of the drive line and the length of the sense line forming the first closed circuit are greater than those forming the second closed circuit, and thus the resistance of the first closed circuit is greater than the second closed circuit.

In an embodiment e.g. as illustrated in FIG. 4, in order to determine the amount of the liquid absorbed in the diaper for the first wetness event, the difference in sense signals between the first location P1 and the second location P2 is determined. As illustrated in FIG. 4, for the first location P1, the resistance of the first closed circuit is composed of the resistance Rs of the length Ls of the sense line S, the resistance Rd of the length Ld of the drive line D, and the resistance Rl of the liquid between the sense and drive lines S and D. It is well known that the resistance of the liquid is much lower than the resistance of the conductive lines, and thus may be omitted when determining the resistance of the closed circuit. Therefore, the first closed circuit at the first location P1 may be considered as having a resistance of Rs+Rd. Similarly, the second closed circuit at the second location P2 may be considered as having a resistance of Rs′+Rd′. With the change of the resistances of the closed circuits between the drive line D and the sense line S, the sense signal (e.g. the current) sensed from the sense line S also changes. In this manner, based on the change of the sense signals for a wetness event, the amount of the liquid exerted from the wetness event can be determined or estimated.

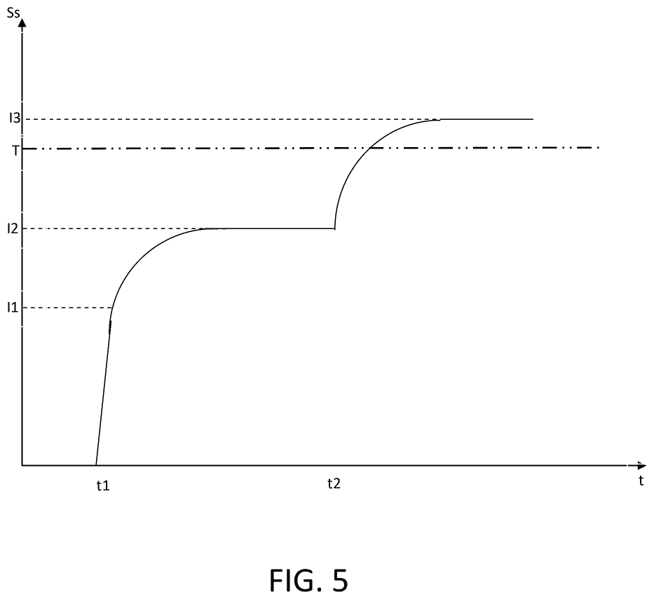

FIG. 5 schematically illustrates a sense signal Ss sensed from the sense line S for wetness events, according to an embodiment of the present disclosure. As an example, FIG. 5 takes current signal I, sensed from the sense line, as the sense signal Ss. Until the time t1 when a first wetness event occurs, no sense signal is sensed by the attachment unit from the sense line because no closed circuit is formed between the drive line D and the sense line S, that is, the sense signal is 0. At the time t1, a first wetness event occurs and a first closed circuit is formed at a first location P1, and thus a current signal I1 is sensed by the unit from the sense line S. The abrupt change of the sense signal from 0 to I1 may be used as indication for the first wetness event. In the context of the present disclosure, an abrupt change of a signal refers to a substantial change (e.g. an increase of at least 30%, at least 50%, at least 80%, or at least 100% of initial value, or a change from zero (0) or a negligible value (e.g. less than a measurement resolution, less than 2 measurement resolutions, less than 3 measurement resolutions, and etc.) to at least a nonnegligible value (e.g. at least several measurement resolutions such as at least 5 measurement resolutions, at least 10 measurement resolutions, at least 20 measurement resolutions, at least 50 measurement resolutions, at least 100 measurement resolutions, and etc), or a change beyond a threshold (either an absolute value threshold or a percentage threshold)) of a signal, such substantial change may occur during a short time period (e.g. a time period shorter than a threshold) such as less than 2 seconds, less than 5 seconds, less than 10 seconds, and etc, e.g. a sharp rise or a jump, ideally a sharp edge. It is understood that, an abrupt change of a signal may be defined by a change threshold and a time threshold, that is, a change of signal will be considered as an abrupt change if the signal changes at least the amount of the change threshold over the time period of the time threshold. But it is also possible for an abrupt change of a signal to be defined by a change threshold, that is, a change of signal will be considered as an abrupt change if the signal changes at least the amount of the change threshold irrespective of time.

With the end of the urination and with the absorption and expansion of the liquid in the diaper, the closed circuit changes gradually from the first closed circuit at the first location P1 to a second closed circuit at a second location P2. Meanwhile, the lengths of the drive line D and the sense line S forming the second closed circuit are gradually decreased to Ld′ and Ls′ respectively, and the resistance of the closed circuit decreases gradually from Rs+Rd to Rs′+Rd′. Consequently, the sense signal increases gradually from I1 until I2, which I2 retains substantially unchanged until the time t2 when a second wetness event occurs. Based on the difference between I1 and I2, the liquid exerted from the first wetness event or the liquid absorbed during the first wetness event can be determined or estimated.

When the second wetness event occurs at t2, the liquid again hits the diaper and further expands and ends in forming a third closed circuit between the drive and sense lines D and S at a third location P3. Similarly, starting from t2, the sense signal increases gradually from I2 until I3, which also retain substantially unchanged until a next wetness event. Based on the difference between I2 and I3, the liquid exerted from the second wetness event can be determined or estimated. Similarly, based on the difference between I1 and I3, the liquid absorbed in the diaper after the second wetness event can be determined or estimated, which can be further used to determine whether the diaper is saturated or not.

The above behavior of the sense signal repeats for each further wetness event, until the diaper reaches saturation. It is understood that the saturation of an absorbent article may refer to a certain amount/volume of liquid in the absorbent article, or a certain degree/extent/percentage the liquid is saturating the absorbent article (i.e. how wet the absorbent article is).

In order to determine the saturation in an absorbent article such as diaper, a threshold T which depends on the liquid capacity of the absorbent article (or the maximum absorption of liquid in the absorbent article, or the maximum amount of liquid that can be absorbed in the absorbent article) is preset or predetermined, thereby the absorbent article may be considered as being saturated when the sense signal is above the threshold T. Further, in response to the saturation of an absorbent article such as a diaper, an alarm may be generated, e.g. in order to request or prompt the change of the absorbent article. As an example, a threshold T is schematically depicted in FIG. 5, in which the diaper is considered as being saturated after the second urination at t2 because the sense signal I3 is above this threshold T.

From the above, it is understood that the threshold T is critical to the determination of saturation in an absorbent article. On the other hand, it is found that even with a same amount of liquid, the absorption and/or expansion of the liquid in a same absorbent article may vary with several factors e.g. the wearing state/manner of the absorbent article on the wearer (such as whether the absorbent article is worn in a loose or tight manner, and/or the orientation of the absorbent article or of the wearer when wetness event occurs, and/or etc.), the movement of the wearer of the absorbent article, and etc. That is, these several factors might impact the signal sensed from the absorbent article and in turn impact the determination of saturation in the absorbent article. Also, it is appreciated that the conductive line(s) provided on an absorbent article may have a characteristic (e.g. resistance) that varies depending on e.g. the associated printing process, and in turn might impact the signal sensed from that particular absorbent article and thus impact the determination of saturation in that particular absorbent article, that is, even with a same amount of liquid, with a same drive signal, and with the same wearing state/manner of absorbent article on a same wearer with the same orientation and movement, the sensed signal may vary from one absorbent article to another because of the characteristics (e.g. resistance) of their respective conductive lines. As such, it is impossible to achieve an accurate determination of saturation by using a fixed threshold. Therefore, in an embodiment of the present disclosure, the saturation in an absorbent article is determined or estimated based on an adjustable threshold, in order to obtain a more accurate determination or estimation. Although the wetness/saturation detection of an absorbent article is described previously herein by using the combination of conductive lines in the absorbent article and an attachment unit, embodiments of the present disclosure are not so limited, and it is appreciated that the threshold adjustment according to the present disclosure may be used with any wetness/saturation detection method, which falls into the spirit and scope of the present disclosure.

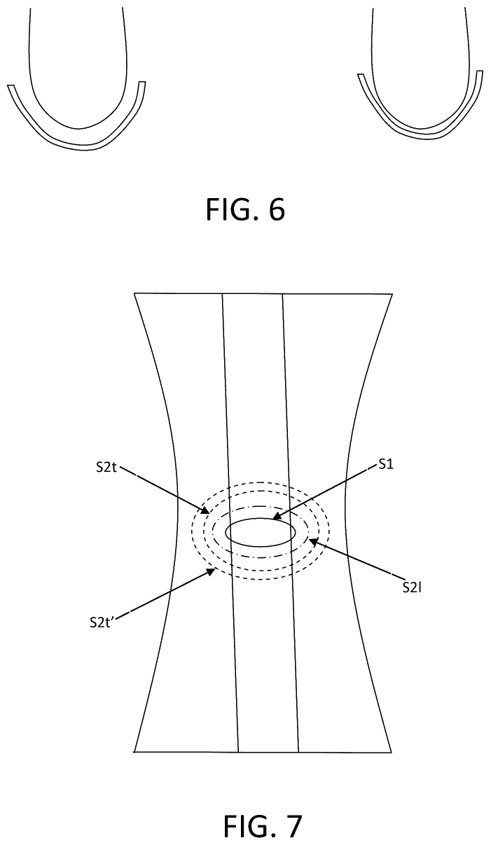

It is understood that, depending on e.g. the personal preference of the wearer or the caregiver who takes care of the wearer, an absorbent article may be worn in different manners/states, e.g. in a either loose or tight manner, by the wearer herself/himself or by a caregiver. FIG. 6 schematically illustrates two wearing states/manner of an absorbent article, a loose state on wearer and a tight state on wearer, according to an embodiment of the present disclosure. As illustrated in FIG. 6, when an absorbent article such as a diaper is worn in a loose manner, the absorbent article is less close to the wearer's body (please refer to the left diagram of FIG. 6), and when an absorbent article is worn in a tight manner, the absorbent article is more close to the wearer's body (please refer to the right diagram of FIG. 6).

As described above, when a person wearing a diaper urinates in the diaper, her/his urine produces at the start of her/his urination a first spot in the diaper that makes at least two parallel conductive lines in the diaper to be connected to each other, and then the urine spot gradually spreads/develops/grows during the urination as the amount of urine increases, and finally become a second bigger spot at the end of this urination. After the end of this urination, the urine spot remain substantially unchanged (e.g. does not spread/develop/grow further) until the person's next urination.

When the diaper is worn in a tight manner, there exists a tighter contact or a greater pressure between the wearer's body and the diaper, which makes the urine travel/spread further more in the diaper and in turn results in a bigger urine spot at the end of the urination, when compared with a loose state of the diaper on the wearer. Further, because of the tighter contact or a greater pressure between the wearer's body and the diaper, the urine spot will spread/develop/grow further a little bit for e.g. another 1-2 minutes in the diaper after the end of the urination, when the diaper is worn in a tight manner.

FIG. 7 schematically illustrates the development of the urine spot in an absorbent article, according to an embodiment of the present disclosure. As illustrated in FIG. 7, a first urine spot S1 (as illustrated as solid line) is produced in an absorbent article at the beginning of an urination (e.g. a first urination) in the absorbent article, regardless of whether the absorbent article is worn in a loose or tight manner. As the amount of urine increases during the urination and as the urine is absorbed and expands/spreads in the absorbent article, a second urine spot S2l (as illustrated as dot-dash line) is produced in the absorbent article when the absorbent article is worn in a loose manner, or a second urine spot S2t (as illustrated as dashed line) is produced in the absorbent article when the absorbent article is worn in a tight manner, both at the end of the urination or shortly after. The second urine spot S2t is bigger than the second urine spot S2l, even though it is the same amount of urine, as illustrated in FIG. 7. Further, when the absorbent article is worn in a tight manner, the urine spot further spreads/develops a little bit for e.g. 1-2 minutes after the end of the urination, thereby producing after the end of the urination a third urine spot S2t′ (also as illustrated as dashed line) that is even bigger than the second urine spot S2t. For the subsequent urination(s), the development of the urine spot as described above will repeat, with the urine spot expanding/spreading from that produced by the immediately preceding urination.

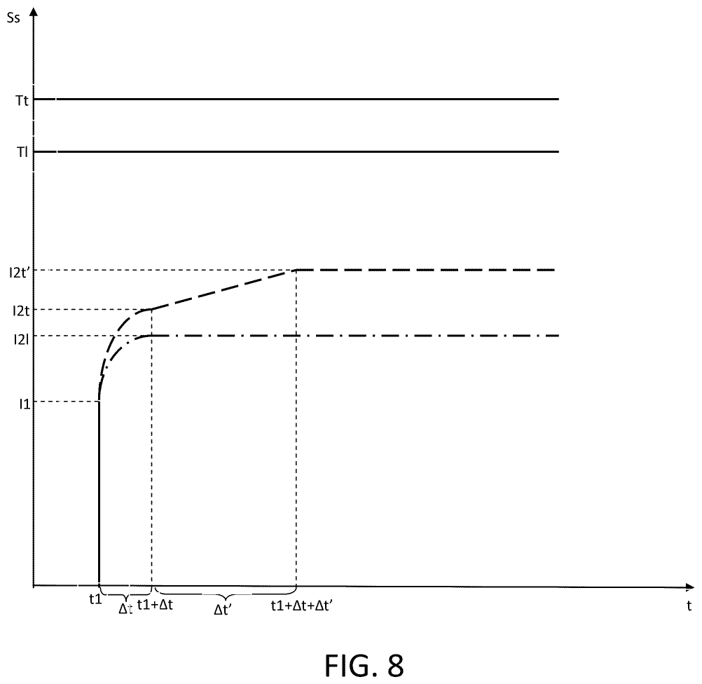

FIG. 8 schematically illustrates the example signal sensed from the absorbent article for the urination as illustrated in FIG. 7, according to an embodiment of the present disclosure. Similar to FIG. 5, FIG. 8 also takes current signal I, sensed from the sense line, as the sense signal Ss. Until the time t1 when the first urination as illustrated in FIG. 7 occurs, no sense signal is sensed from the absorbent article because no closed circuit is formed between the conductive lines in the absorbent article, that is, the sense signal is 0. At the time t1, the urination occurs and the first spot S1 is produced in the absorbent article which connects two conductive lines in the absorbent article, and thus a current signal I1 is sensed from the absorbent article. The abrupt change of the sense signal from 0 to I1 may be used as indication for the start of the wetness event.

The urination lasts a certain amount of time Δt e.g. 10 seconds, during which the urine spot gradually spreads/develops from S1 to S2l or S2t in the absorbent article depending on the wearing manner/state (i.e. tight or loose) of the absorbent article on the wearer. Correspondingly, during the urination Δt, the sense signal Ss gradually increases from I1 and achieves I2l or I2t at the end of the urination at time t+Δt, depending on the wearing manner/state of the absorbent article on the wearer. As can be seen from FIG. 8, I2t is greater than I2l. Further, as described above, when the absorbent article is worn in a tight manner, the urine spot continues to spread/develop for another amount of time Δt′ e.g. 1-2 minutes after t+Δt (i.e. after the end of the urination) further to S2t′, which results in the sense signal Ss further increasing after t+Δt (i.e. after the end of the urination) from I2t to e.g. I2t′ which then remain substantially constant. On the contrary, when the absorbent article is worn in a loose manner, the urine spot will remain substantially unchanged after t+Δt i.e. after the end of the urination, and therefore the sense signal Ss will remain substantially constant after t+Δt i.e. after the end of the urination.

Based on the above, the wearing manner/state (i.e. tight or loose) of an absorbent article on a wearer may be determined based on the sharp rise (e.g. the first sharp rise) and/or the behavior thereafter in the sense signal sensed from the absorbent article worn on the wearer, and accordingly the threshold for the saturation of the absorbent article worn on the wearer may be adjusted, in an embodiment of the present disclosure.

As can be seen from FIG. 8, from the behavior of the sense signal after the end of urination (e.g. after the end of the first urination, such as after t+Δt as illustrated in FIG. 8), e.g. the difference between I2t′ and I2t as illustrated in FIG. 8, it is possible to determine whether an absorbent article is worn in a loose or tight manner, and/or the tightness (how tight) or looseness (how loose) of the absorbent article on the wearer. In particular, an absorbent article may be considered as being worn in a loose manner when there is no substantial increase in the sense signal after the end of urination, according to an embodiment of the present disclosure. On the other hand, when there exists increase in the sense signal after the end of urination, an absorbent article may be considered as being worn in a tight manner, and the more the increase in the sense signal after the end of urination is, the tighter the absorbent article is worn. In an embodiment of the present disclosure, the wearing manner/state (i.e. tight or loose) of an absorbent article on a wearer may be determined based on the behavior of the sense signal after the end of urination (e.g. the increase in the sense signal until being substantially constant, such as the increase from I2t to I2t′ as shown in FIG. 8), and the threshold for the saturation of the absorbent article worn may be adjusted accordingly.

Alternatively or in addition, the wearing manner/state (i.e. tight or loose) of an absorbent article on a wearer may be determined based on the behavior of the sense signal during urination (e.g. the increase in the sense signal from the start of the urination (i.e. when the urination occurs, and at the sharp rise of the sense signal) to the end of urination, such as the increase from I1 to I2l or I2t as shown in FIG. 8), and the threshold for the saturation of the absorbent article worn may be adjusted accordingly. As described above, during an urination (e.g. a first urination), the sense signal gradually increases from an initial value (e.g. 0 for a first urination or a previous end value at the end of the immediately preceding urination) at the beginning of the urination (i.e. at the sharp rise of the signal) to an end value (e.g. I2l or I2t as illustrated in FIG. 8) at the end of the urination, depending on the wearing manner/state of the absorbent article on the wearer. That is, the end value at the end of an urination, and/or the difference between the initial and end values of an urination may reflect the wearing manner/state (i.e. tight or loose) of the absorbent article on the wearer, and thus may be used to adjust the threshold for saturation of the absorbent article worn on the wearer, in an embodiment of the present disclosure.

As described above, in order to determine whether an absorbent article is worn in a loose or tight manner or to determine the tightness or looseness of an absorbent article on the wearer, the behavior of the sense signal Ss after the end of urination may be determined. According to an embodiment of the present disclosure, the end of urination may be determined by using a predetermined amount of time e.g. Δt as illustrated in FIG. 8. That is, the end of urination may be considered at Δt after the start of urination (the time t1), i.e. at t1+Δt. As an example, the predetermined amount of time e.g. Δt may be 10 or 20 seconds. Alternatively or in addition, the end of urination may be determined by locating the inflection point of the increase in the sense signal Ss after the start of urination (t1). As an example, the urination may be considered as ending at the time where the inflection point occurs in the sense signal Ss after the start of urination (t1), or ending at the time t1+F*(the time interval from t1 to the time of the inflection point), wherein F is a configurable factor which may be e.g. 2, 1.5, etc. It is understood that an inflection point of a signal can be determined or calculated by using a variety of methods.

Further, the threshold T for saturation in an absorbent article may be adjusted based on the determination of the wearing manner/state of the absorbent article on the wearer, according to an embodiment of the present disclosure. In particular, the threshold T for saturation in an absorbent article may be increased (e.g. to Tt as illustrated in FIG. 8) when the absorbent article is worn in a tight manner, and the amount of the increase in the threshold T may depend on the tightness (i.e. how tight) of the absorbent article on wearer. Alternatively or in addition, the threshold T for saturation in an absorbent article may be decreased (e.g. to Tl as illustrated in FIG. 8) when the absorbent article is worn in a loose manner, and the amount of the decrease in the threshold T may depend on the looseness (how loose) of the absorbent article on wearer.

As mentioned above, the conductive line(s) provided on an absorbent article for wetness and/or saturation detection may have characteristics (e.g. electrical characteristics such as resistance) that vary depending on its manufacturing, e.g. depending on the associated printing process, the printing material, and etc., which in turn might impact the signal sensed from the absorbent article and thus impact the determination of saturation in the absorbent article. It is appreciated that in some cases the resistances of the conductive lines provided in an absorbent article may vary a lot depending on its manufacturing such as printing process, and printing material, e.g. sometimes it may vary from 50 k ohms up to 400 k ohms, as an example.

According to an embodiment of the present disclosure, the threshold for saturation in an absorbent article may be adjustable in consideration of the varying characteristics e.g. resistance of conductive lines. In this regard, a baseline that reflects characteristics of the conductive line(s) in an absorbent article may be determined at a certain instance of time e.g. at or after a wetness event, and the threshold for saturation in the absorbent article may be adjusted based on the baseline as determined, in an embodiment of the present disclosure. As an example, the baseline may be determined at the time when a wetness event starts, at the time when a wetness event ends (e.g. 5 seconds after the wetness event starts), at the inflection point of the sense signal Ss after a wetness event starts, or a predetermined time period after a wetness event starts or ends or after the inflection point.

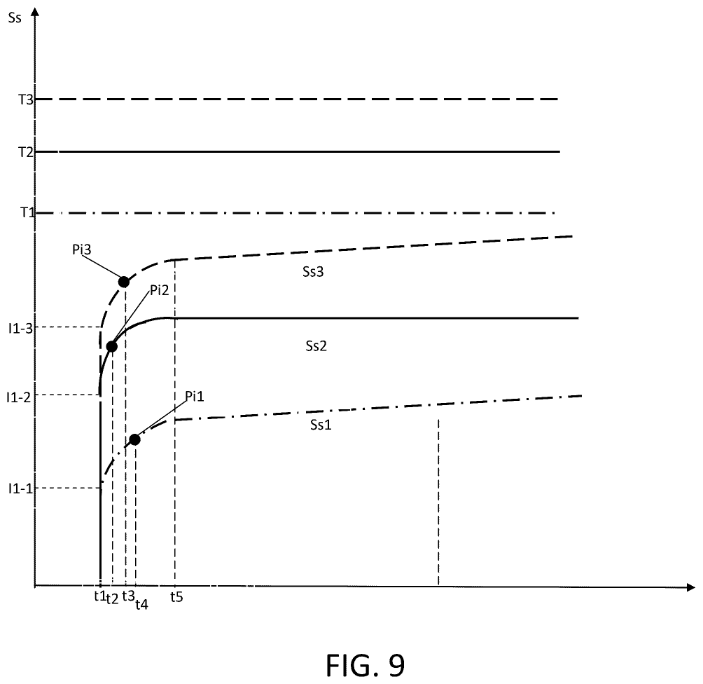

As an example, FIG. 9 schematically illustrates how to determine a baseline and how to adjust threshold based on the baseline, according to an embodiment of the present disclosure. Similarly to FIG. 8, an example signal sensed from an absorbent article upon/after a wetness event (e.g. an urination) as illustrated in FIG. 7 is schematically illustrated in FIG. 9. Until the time t1 when a first wetness event e.g. a first urination as illustrated in FIG. 7 occurs, no sense signal is sensed from the absorbent article because no closed circuit is formed between the conductive lines in the absorbent article, that is, the sense signal is 0. At the time t1, a first wetness event e.g. an urination occurs and the first spot S1 is produced in the absorbent article which connects two conductive lines in the absorbent article, and thus a current signal I1 (I1-1, I1-2, I1-3) is sensed from the absorbent article. The abrupt change of the sense signal from 0 to I1 or a change of the sense signal beyond a threshold may be used as indication for the start of the wetness event. Next the sensed signal Ss develops/behaves in the same manner as described above e.g. in connection with FIG. 8, it keeps going up and eventually reaches a threshold for saturation.

As mentioned above, the sensed signal Ss depends on the resistance of the closed circuit formed after a wetness event, and in particular, depends on the resistance of the conductive lines forming the closed circuit. In FIG. 9, three sensed signal Ss (Ss1, Ss2, and Ss3) are plotted schematically for three absorbent articles with conductive lines in a same arrangement, illustrating how the difference in characteristics e.g. resistance of conductive lines provided in the three absorbent articles impacts the sensed signal. As an example, the sensed signal Ss at the time when the first wetness event starts (i.e. t1) may reflect the corresponding characteristics e.g. resistance of conductive liens and thus may be used as baseline to adjust the threshold for saturation. As illustrated in FIG. 9, with all other conditions (e.g. the drive signal, the amount of the wetness event, the wearing state of the absorbent article, and etc.) being the same, the sensed signal Ss1 has a current signal I1-1 at t1 for a first absorbent article A1, the sensed signal Ss2 has a current signal I1-2 at t1 for a second absorbent article A2, while the sensed signal Ss3 has a current signal I1-3 at t1 for a third absorbent article A3, and I1-3>I1-2>I1-1, because the resistance of conductive lines in A1 is greater than that in A2, which in turn is greater than that in A3. Then the current signal (e.g. I1-1, I1-2, I1-3) at t1 may be used as baseline to adjust the threshold for saturation, in order to take into account the difference in characteristics e.g. resistance of conductive lines, and thus also the particular manufacturing (e.g. printing) of the conductive lines. In the example as illustrated in FIG. 9, the high value of I1-3 implies a less resistance of conductive lines in the absorbent article A3, and thus a threshold for saturation T3 shall be adjusted or set to a high value, while the low value of I1-1 implies a high resistance of conductive lines in the absorbent article A1, and thus a threshold for saturation T1 shall be adjusted or set to a low value. It is appreciated that the baseline may be determined at other time point, e.g. t2 (corresponding to the inflection Pi2 of the signal Ss2), t3 (corresponding to the inflection Pi3 of the signal Ss3), t4 (corresponding to the inflection point Pi1 of the signal Ss1), or t5 (corresponding to the end of the urination).

It is to be noted that t1, the time when a first wetness event starts, is only an example instance of time to determine the baseline, and it is conceivable for those skilled in the art to determine the baseline at an instance of time after a wetness event other than t1, e.g. at the time when the wetness event ends, at the inflection point of the sensed signal after the wetness event, or a predetermined time period (e.g. 1 second, 2 seconds, 3 seconds) after any of the above-mentioned instance of times. It is also appreciated that the determination of baseline and the adjustment of threshold can be conducted for every wetness event, so that the threshold for saturation can be adjusted dynamically during the whole period when a wearer wears an absorbent article.

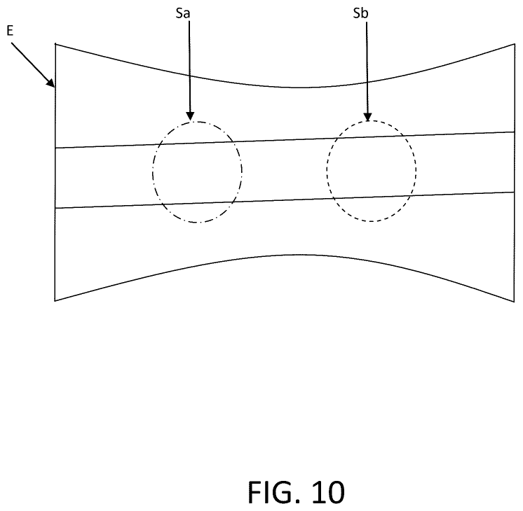

As mentioned above, the angle that a wetness event occurs in relative to an absorbent article might also impact the signal sensed from the absorbent article and in turn impact the determination of saturation in the absorbent article. As an example, FIG. 10 schematically illustrates two example wetness (e.g. urination) spots Sa and Sb produced when a wetness event occurs with different angles in relative to an absorbent article with all other conditions (e.g. the wetness amount, the resistance of conductive lines, and etc.) being the same. As an example and as illustrated in FIG. 10, when a person conducts a wetness event e.g. urination while lying down e.g. on her/his back, the wetness spot Sb will be farther away from the front waist edge E of the absorbent article, and thus the closed circuit as formed will have a higher resistance and the sensed current signal will be lower. On the other hand, when a person conducts a wetness event e.g. urination while sitting, the wetness spot Sa will be closer to the front waist edge E of the absorbent article, and thus the closed circuit as formed will have a lower resistance and the sensed current signal will be higher. It is understood that the threshold for saturation may be adjusted accordingly, e.g. it may be adjusted to a high value for sitting position while to a low value for lying position, in an embodiment of the present disclosure.

It is understood that the angle that a wetness event occurs in relative to an absorbent article has a similar effect as the different characteristics e.g. resistance of conductive lines, and thus the baseline determined e.g. in FIG. 9 can also reflects the angle that a wetness event occurs in relative to an absorbent article. Therefore, in an embodiment of the present disclosure, the baseline as determined e.g. according to FIG. 9 may be used to adjust the threshold for saturation, in order to take into account the angle that a wetness event occurs in relative to an absorbent article. Also, it is appreciated that the angle that a wetness event occurs in relative to an absorbent article depends on the orientation of the absorbent article or its wearer when the wetness event occurs in the absorbent article, and the orientation of an absorbent article or its wearer can be determined with aid of an orientation means (e.g. accelerator, gyroscope, and etc.) provided in the attached unit. Therefore, in an embodiment of present disclosure, the threshold for saturation may be adjusted based on the measurement from the orientation unit provided in the attachment unit, in order to take into account the angle that a wetness event occurs in relative to an absorbent article.

As mentioned above, for the same amount of liquid, the absorption and expansion of the liquid in a same absorbent article may vary with the movement of the wearer of the absorbent article. In particular, when there is more motion, e.g. when the wearer of an absorbent article is running or walking, the liquid e.g. urine in the absorbent article will spread or develop more. On the other hand, when there is no motion, e.g. when the wearer of an absorbent article is sitting or sleeping, the liquid e.g. urine in the absorbent article will stay stationary. In an embodiment of the present disclosure, the movement may be detected periodically (e.g. every 20 seconds, etc.). As a non-limiting example, the movement may be determined based upon the change in orientation of the wearer or the absorbent article worn thereon measured periodically (e.g. every 20 seconds, etc.).

Therefore, according to an embodiment of the present disclosure, a motion detector and/or an orientation detector is provided in the attachment unit that is configured to be attached to an absorbent article and to operate in combination with the spaced-apart conductive lines in the absorbent article for moisture detection and estimation of the absorbent article. The motion/orientation detector is configured to determine the motion/orientation of the wearer of the absorbent article, based on which the threshold of saturation in the absorbent article may be adjusted, according to an embodiment of the present disclosure. As an example, when compared with there existing no movement (or no change in orientation) of the wearer of an absorbent article, the threshold T of saturation in the absorbent article may be increased if there is movement of the wearer, and the more the movement there is, the more the threshold is increased. Alternatively or in addition, when compared with there existing movement of the wearer of an absorbent article, the threshold T of saturation in the absorbent article may be decreased if there is less movement of the wearer, and the less the movement there is, the more the threshold is decreased.

As mentioned above, in operation an attachment unit (e.g. pod) is in general attached to an absorbent article on its front waist portion, and in order to determine the saturation in the absorbent article a measurement is conducted in the closed circuit formed by the attachment unit (e.g. pod) attached to the absorbent article, the conductive lines provided on/in the absorbent article, and the liquid that connects the conductive lines. It is also understood that the change in the body orientation of the wearer of an absorbent article may move the liquid in the absorbent article. Therefore, according to an embodiment of the present disclosure, the change in the body orientation (e.g. forward motion) of the wearer of an absorbent article may be taken into account to adjust the threshold for determining the saturation in the absorbent article. To this end, there may provide e.g. in the attachment unit (e.g. pod) that is configured to be releasably attached to an absorbent article an orientation unit that is configured to determine the body orientation of the wearer of the absorbent article with the attachment unit attached thereon.

Let's consider a case in which a wearer of an absorbent article is in a certain position (e.g. is lying down), and experiences a wetness event e.g. urinates a certain amount of liquid. If the wearer moves her/his body forward (e.g. sits up straight) after the wetness event e.g. her/his urination, the liquid will be pushed forward towards the front waist edge of the absorbent article and thus towards the attachment unit (e.g. pod) and thus go higher, which results in a reduced resistance of the closed circuit and thus a higher signal being sensed. In consideration of the increase in the sensed signal, caused by the wearer's forward motion, with respect to the same amount of liquid, the threshold for determining the saturation in the absorbent article may be increased accordingly, in an embodiment of the present disclosure. Similarly, when a wearer of an absorbent article moves backwards (e.g. from sitting straight to lying down) which will pull back the liquid in the absorbent article a little and result in a lower signal, the threshold for determining the saturation in the absorbent article may be decreased accordingly, in an embodiment of the present disclosure.

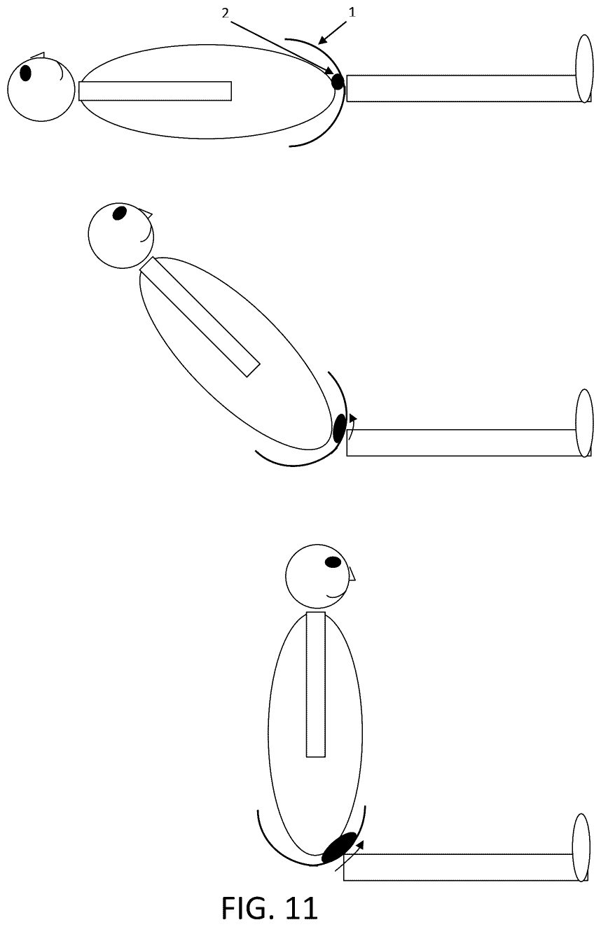

As an example, FIG. 11 schematically illustrates an example movement of an absorbent article's wearer from lying down to sitting up. As illustrated in FIG. 11, at first a wearer of an absorbent article 1 is lying back and urinates in her/his lying position, producing a wetness spot 2 in the absorbent article 1. After her/his urination, the wearer sits forward e.g. sits up, which will push the liquid (and thus the wetness spot 2) in the absorbent article forward (as illustrated by arrows in FIG. 11) and in turn will result in a higher sensed signal with respect to a same amount of liquid. Therefore, the threshold for determining the saturation in the absorbent article may be increased accordingly, in an embodiment of the present disclosure.

On the other hand, as another example, a wearer of an absorbent article may be sitting straight and urinates in her/his sitting position. After her/his urination, the wearer moves her/his body backwards e.g. lies down, which will pull the liquid in the absorbent article back a little more and in turn will result in a lower sensed signal with respect to a same amount of liquid. Therefore, the threshold for determining the saturation in the absorbent article may be decreased accordingly, in an embodiment of the present disclosure.

It is possible for a wearer of an absorbent article experiences more than one changes in her/his body orientation (e.g. more than one forward motion, such as sit up and lie back, then sit up again and lie back again, and then sit up again) after a wetness event e.g. urination. Therefore, according to an embodiment of the present disclosure, the total change in the body orientation (e.g. forward motions and/or the total backward motions) after the wetness event e.g. urination may be counted, combined (e.g. accumulated), and used to adjust accordingly the threshold for determining the saturation in the absorbent article. In an embodiment of the present disclosure, only forward motions may be counted and accumulated for the total change in body orientation that is used to adjust the threshold for saturation, that is, the total change in body orientation may not take into account the backward motions.

It is to be noted that the liquid will be fully absorbed in the absorbent article after a certain amount of time following a wetness event such as urination. It is further to be noted that the wearer's motion such as change in body orientation (e.g. forward motion and backward motion) does not move the liquid anymore when the liquid is already fully absorbed in the absorbent article. Therefore, the wearer's motion may be taken into account to adjust the threshold, but only for a certain period after a wetness event e.g. her/his urination. That is, the (total) motion such as the (total) change in body orientation (e.g. forward and/or backward motion) of the wearer will be counted, and combined (e.g. accumulated) for only a certain period (e.g. 30 minutes) after a wetness event e.g. her/his urination.

It is also noted that, as more and more liquid is absorbed in an absorbent article over time, the effect of the wearer's motion on the spread of liquid in the absorbent article will gradually change (e.g. reduce) over time. Therefore, according to an embodiment of the present disclosure, different weights may be assigned to the wearer's motion during different periods after a wetness event e.g. urination. As an example, a highest weight (e.g. 100%) may be assigned to the wearer's motion within the first 10 minutes after a wetness event e.g. her/his urination, a lower weight (e.g. 30%) may be assigned to the wearer's motion within the following 10 minutes, and an even lower weight (e.g. 0%) may be assigned to the wearer's motion after the first 20 minutes following a wetness event. In this way, the threshold for determining the saturation in an absorbent article may be adjusted accordingly in response to the wearer's motion at different time. Further, the total motion such as the total change in body orientation e.g. forward and/or backward motion of the wearer may take into account the different weight assigned to her/his motions at different times when being used to adjust the threshold for determining the saturation in an absorbent article.

There exist other motions of an absorbent article's wearer without any change in her/his body orientation, which also may move the liquid in the absorbent article. As an example, the wearer may sit up and/or sit down and/or stand up without changing her/his body orientation, where there exists a velocity vector in a vertical direction.

It is understood that when a wearer of an absorbent article sits down with a vertical velocity, such vertical motion might push the liquid in the absorbent article to make it expand/spread in the absorbent article, which in turn causes a higher signal to be sensed with respect to a same amount of liquid in the absorbent article. Accordingly, the threshold for determining the saturation in the absorbent article may be increased accordingly, in an embodiment of the present disclosure.

It is to be noted that, when the vertical velocity is too small, the effect of such vertical motion on the spread of liquid in the absorbent article may be negligible. Therefore, a threshold for velocity vector is preset, and the threshold for determining the saturation in an absorbent article will be adjusted only when the vertical velocity vector is above that preset threshold, according to an embodiment of the present disclosure.

It is possible for a wearer of an absorbent article experiences more than one vertical motions without change in body orientation (e.g. stand up, walk around, and then come sit down,) after a wetness event e.g. urination. Therefore, according to an embodiment of the present disclosure, the total vertical motions without change in body orientation (above the preset threshold for velocity vector) may be counted, accumulated, and used to adjust accordingly the threshold for determining the saturation in the absorbent article.

The invention claimed is:

1. A method for detecting the saturation of an absorbent article, comprising:
detecting a sense signal from the absorbent article, and
comparing the detected sense signal to a saturation threshold,
wherein the saturation threshold is adjustable.

2. The method according to claim 1, wherein the saturation threshold is automatically adjusted based on tightness of the absorbent article on its wearer's body.

3. The method according to claim 2, further comprising:
determining the tightness of the absorbent article on its wearer's body, and
adjusting the saturation threshold based on the determined tightness.

4. The method according to claim 1, wherein the saturation threshold is automatically adjusted based on amount of the movement of the wearer of the absorbent article.

5. The method according to claim 4, further comprising:
determining the movement of the wearer of the absorbent article during a certain time period after a wetness event, and
adjusting the saturation threshold based on the determined movement of the wearer.

6. The method according to claim 5, wherein the adjusting the saturation threshold comprises:
continuously adjusting the saturation threshold based on the total amount of movements of the wearer of the absorbent article over time during the certain time period after the wetness event.

7. The method according to clam 5, wherein the adjusting the saturation threshold comprises:
continuously adjusting the saturation threshold based on the weighted sum of movements of the wearer of the absorbent article over time during the certain time period after the wetness event.

8. The method according to claim 7, wherein the weight for the weighted sum decreases over time.

9. The method according to claim 4, wherein the movement is determined based upon the change in orientation of the absorbent article measured periodically.

10. The method according to claim 4, wherein the movement of the wearer of the absorbent article is detected by a movement detector provided in an attachment unit that is configured to be attached to the absorbent article and to apply the drive signal and sense the sense signal.

11. The method according to claim 1, wherein the saturation threshold is automatically adjusted based on the change in the sense signal detected after the end of a wetness event in the absorbent article.

12. The method according to claim 11, wherein the end of the wetness event is determined based on the start of the wetness event and one of a predetermined fixed time interval or the inflection point of the sense signal after the start of the wetness event.

13. A method for detecting a wearing state of an absorbent article on its wearer, the method comprising:
determining a sense signal from the absorbent article after the end of a wetness event in the absorbent article; and
determining the wearing state of the absorbent article on its wearer based on the determined sense signal.

14. The method according to claim 13, wherein the absorbent article is determined as being tight on its wearer when the determined sense signal continues to increase after the end of the wetness event.

15. The method according to claim 13, wherein the end of the wetness event is determined based on the start of the wetness event and one of a predetermined fixed time interval or the inflection point of the sense signal after the start of the wetness event.
